(19) United States
(12) Patent Application Publication
(10) Pub. No.: US 2010/0298580 A1
(43) Pub. Date: Nov. 25, 2010

(54) Process for the Preparation of 2H-Chromene-3-Carbamate Derivatives

(75) Inventors: Brian Broadbelt, S. Mamede do Coronado (PT); Alexander Beliaev, Mindelo (PT); David Alexander Learmonth, Alfena (PT)

Correspondence Address: CONLEY ROSE, P.C., 5601 GRANITE PARKWAY, SUITE 750, PLANO, TX, 75024, US

(73) Assignee: BIAL - PORTELA & CA, S.A., S. Mamede do Coronado (PT)

(21) Appl. No.: 12/742,738

(22) PCT Filed: Nov. 13, 2008
(86) PCT No.: PCT/PT2008/000048

§ 371 Date: Aug. 9, 2010

Related U.S. Application Data
(60) Provisional application No. 60/987,467, filed on Nov. 13, 2007.
(60) Provisional application No. 61/085,927, filed on Aug. 4, 2008.

(51) Int. Cl. C07D 311/58

(52) U.S. Cl. 549/404; 549/405; 549/407

(57) ABSTRACT

Compounds of formula I, V, VI and II, and processes for their preparation,

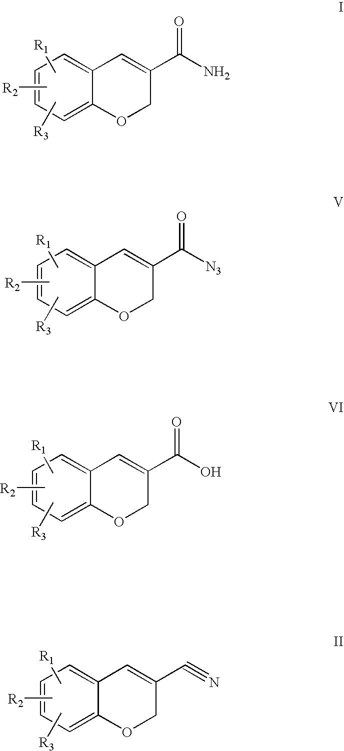

wherein R₁, R₂ and R₃ are the same or different and signify hydrogens, halogens, alkyl, alkyloxy, hydroxy, nitro, alkylcarbonylamino, alkylamino or dialkylamino group; and R₄ is alkyl or aryl. There is also provided a process for preparing a compound of formula B from the compounds of formula V and I.

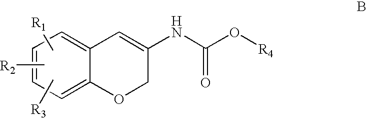

### CROSS-REFERENCE TO RELATED APPLICATIONS

This application is a filing under 35 U.S.C. 371 of International Application No. PCT/PT2008/000048 filed Nov. 13, 2008, entitled “Process for the Preparation of 2H-Chromene-3-Carbamate Derivatives,” claiming priority of U.S. Provisional Patent Application Nos. 60/987,467 filed on Nov. 13, 2007 and 61/085,927 filed on Aug. 4, 2008, which applications are incorporated by reference herein in their entirety.

### BACKGROUND OF THE INVENTION

The present invention relates to an improved process for preparing intermediates useful in the synthesis of peripherally-selective inhibitors of dopamine-β-hydroxylase and novel intermediates.

(R)-5-(2-Aminoethyl)-1-(6,8-difluorochroman-3-yl)-1,3-dihydroimidazole-2-thione hydrochloride (the compound of formula P, below) is a potent, non-toxic and peripherally selective inhibitor of DβH, which can be used for treatment of certain cardiovascular disorders. Compound P is disclosed in WO2004/033447, along with processes for its preparation.

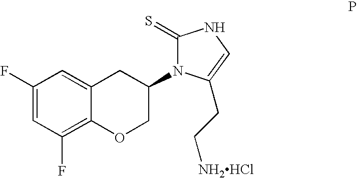

The process disclosed in WO2004/033447 involves the reaction of (R)-6,8-difluorochroman-3-ylamine hydrochloride (the structure of (R)-6,8-difluorochroman-3-ylamine is shown below as compound Q), [4-(tert-butyldimethylsilanyloxy)-3-oxobutyl]carbamic acid tert-butyl ester and potassium thiocyanate.

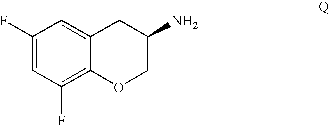

(R)-6,8-difluorochroman-3-ylamine (compound Q) is a key intermediate in the synthesis of compound P. The stereochemistry at the carbon atom to which the amine is attached gives rise to the stereochemistry of compound P, so it is advantageous that compound Q is present in as pure a form as possible. In other words, the R enantiomer of compound Q should be in predominance, with little or no S enantiomer present.

### SUMMARY OF THE INVENTION

Advantageous processes for preparing an intermediate useful in the synthesis of compound P have now been found. The intermediate is a compound having the formula B.

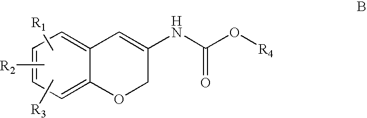

The advantageous processes involve conversion of a compound of formula VII:

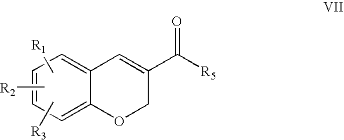

to the compound of formula B, wherein R₄ is alkyl or aryl and R₅ is —N₃ or —NH₂.

One process involves converting a carboxylic azide (i.e. the compound of formula VII in which R₅ is —N₃) to the compound of formula B. The carboxylic azide may be prepared from the corresponding carboxylic acid. The corresponding carboxylic acid may be prepared from the corresponding carbonitrile. The precursor to the corresponding carbonitrile may be produced from a corresponding phenol compound.

Another process involves converting an amide (i.e. the compound of formula VII in which R₅ is —NH₂) to the compound of formula B. The amide may be prepared from the corresponding carbonitrile. The carbonitrile may be prepared from the corresponding aldehyde. The precursor to the aldehyde may be produced from a corresponding phenol compound.

Thus, in its broadest aspect, the present invention provides a process for preparing a compound of formula B comprising converting a compound of formula VII to the compound of formula B:

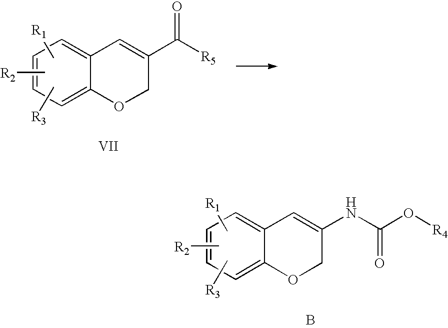

wherein R₁, R₂ and R₃ are the same or different and signify hydrogens, halogens, alkyl, alkyloxy, hydroxy, nitro, alkylcarbonylamino, alkylamino or dialkylamino group; R₄ is alkyl or aryl; and R₅ is —N₃ or —NH₂, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine. In an embodiment, R₅ is —N₃. Alternatively, R₅ is —NH₂. Suitably, the conversion comprises a rearrangement. When R₅ is —N₃, the rearrangement may comprise a Curtius-type rearrangement. When R₅ is —NH₂, the rearrangement may comprise a Hoffman-type rearrangement.

### DETAILED DESCRIPTION

According to one aspect of the present invention, there is provided a process for preparing a compound of formula B:

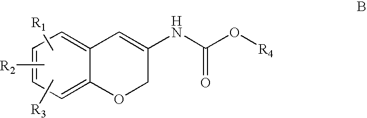

which process comprises converting a compound of formula I

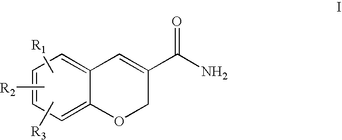

to the compound of formula B, wherein R₁, R₂ and R₃ are the same or different and signify hydrogens, halogens, alkyl, alkyloxy, hydroxy, nitro, alkylcarbonylamino, alkylamino or dialkylamino group; and R₄ is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

In an embodiment, at least one of R₁, R₂ and R₃ is fluorine. In another embodiment, compound I has the following formula IA:

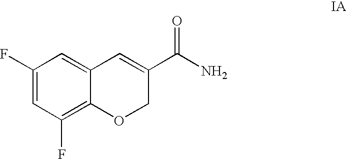

In an embodiment, R₄ is C₁ to C₄ alkyl. Optionally, R₄ is methyl, ethyl or t-butyl. Preferably, R₄ is methyl. In an alternative embodiment, R₄ is benzyl.

In an embodiment, the process is depicted as follows.

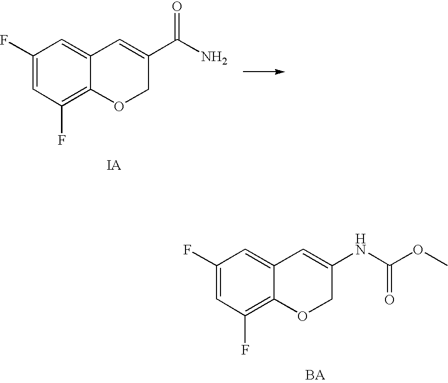

The conversion of I to B may comprise effecting a rearrangement of the amide to form the carbamate, for example a Hoffman rearrangement. The rearrangement may be carried out in the presence of a hypohalite, such as hypochlorite, and an alcohol of the formula R₄OH, where R₄ has the same meanings as given above. Suitably, R₄ is methyl. The hypohalite is typically an alkali metal salt of hypochlorite, for example sodium hypochlorite. Hypohalites other than hypochlorites, for example hypobromites, may also be used in the rearrangement. Suitably, the conversion of I to B comprises rearrangement in the presence of sodium hypochlorite and methanol.

In an embodiment, the compound I and alcohol R₄OH may be stirred at a temperature less than about 10° C. most preferably less than 5° C. whereupon an aqueous solution of alkali metal hypochlorite, typically sodium hypochlorite, is charged at such a rate as to maintain the internal temperature below 10° C. The reaction mass may then be stirred at 5° C. for a period of time typically 30 minutes. The reaction mass comprising the N-chloroamide intermediate should then be made alkaline by addition of a solution of a base such as an alkali metal hydroxide, typically sodium hydroxide, charged to the reaction mass at such a rate as to maintain the internal temperature below about 10° C. The temperature of the reaction mass may then be maintained below 10° C. for a period of time typically about 30 minutes, before adjusting the temperature of the reaction mass to a temperature ranging from about 20° C. to about 30° C., typically 25° C. This temperature may then be maintained for a period of time ranging from about 15 hours to about 30 hours, typically about 20 hours to about 25 hours whereupon the reaction mass is then adjusted to a temperature below 10° C., typically about 5° C., before charging water, and maintaining the temperature of the resulting suspension at about 5° C., for at least 1 hour. The product can then be filtered and washed with aqueous methanol (typically 1:1, H₂O:MeOH) and dried under vacuum compound B as a white microcrystalline solid.

The product of the conversion of B to I may be purified, for example by recrystallisation. The recrystallisation may be effected in the presence of a mixture of water and an alcohol such as 2-propanol.

According to another aspect of the present invention, there is provided a process for preparing a compound of formula I, as defined above. The process involves converting a compound of formula II:

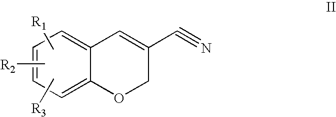

to the compound of I, wherein R₁, R₂, and R₃ have the same meanings as given above.

In an embodiment, at least one of R₁, R₂ and R₃ is fluorine. In an embodiment, the compound of formula II has the formula IIA:

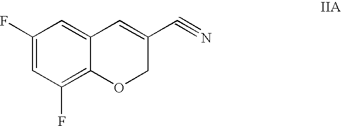

The conversion of II to I may involve hydrolysis in the presence of a mineral acid and an organic acid. The mineral acid may be sulfuric acid. The organic acid may be acetic acid. The reaction medium may be a mixture of acetic acid and sulfuric acid.

In an embodiment, the mineral acid is added to compound II, in organic acid, with stirring at a temperature ranging from about 15° C. to about 25° C., typically about 20° C. The temperature of the reaction mass may then be increased to a temperature ranging from about 80° C. to about 110° C., typically about 100° C., and the temperature maintained for a period of time typically about 45-90, for example 60, minutes. The temperature of the reaction mass may then be decreased to a temperature ranging from about 25° C. to about 35° C., typically about 30° C. and aqueous alcohol such as aqueous isopropanol (typically 2:1, water:IPA) charged to the reaction mass over a period of time typically about 20 minutes. The temperature of the reaction mass may then be decreased to a temperature below 10° C., typically 5° C., and maintained at this temperature for at least 2 hours. The product can then be filtered and the filter cake washed with further aqueous alcohol solution such as aqueous isopropanol (typically 2:1, water:IPA). The product may then be dried under vacuum at around 40° C. to yield compound I.

In an embodiment, the process is depicted as follows.

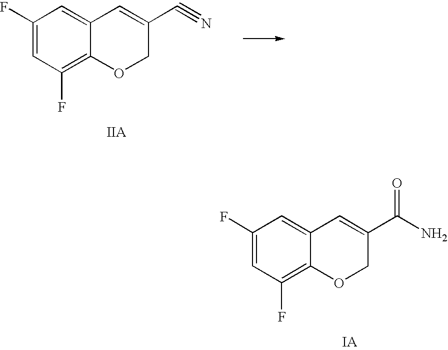

The compound of formula II, as defined above, may be prepared by converting a compound of the formula III:

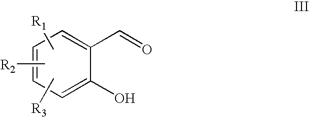

to the compound of formula II, wherein R₁, R₂ and R₃ have the same meanings as given above.

In an embodiment, at least one of R₁, R₂ and R₃ is fluorine. In an embodiment, the compound of formula III has the formula IIIA:

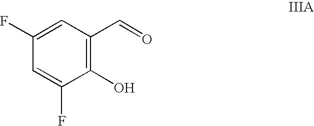

The conversion of III to II involves a cyclocondensation reaction, such as reacting the compound of formula III with acrylonitrile in the presence of 1,4-diazabicyclo[2.2.2] octane (DABCO). The reaction mixture may be heated to an elevated temperature, for example a temperature ranging from 50° C. to 90° C., preferably from 60° C. to 80° C., more preferably around 70° C. The reaction may be carried out in neat acrylonitrile or using a solvent such as acetonitrile or DMF.

The compound of formula III may be prepared by converting a compound of formula IV:

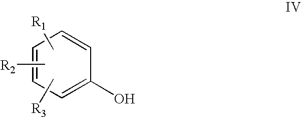

to the compound of formula III, wherein R₁, R₂ and R₃ have the same meanings as given above. In an embodiment, at least one of R₁, R₂ and R₃ is fluorine. In an embodiment, the compound of formula I has the formula IVA:

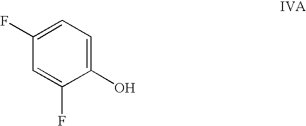

The conversion of IV to III may involve reacting the compound of formula IV with a formylating agent. In an embodiment, the reaction is carried out in the presence of an acid. The formylating agent may be hexamethylenetetramine and the acid may be trifluoroacetic acid.

After addition of the formylating agent, the temperature of the reaction mixture may be raised, for example to a temperature ranging from 60° C. to 100° C., preferably from 70° C. to 90° C., more preferably to a temperature of around 80° C. This temperature may be maintained for a period of time for example of at least 60 minutes. The temperature of the reaction mixture may be further raised to a temperature ranging from about 90° C. to about 130° C., preferably from about 100° C. to about 120° C., more preferably to a temperature of about 115° C. The reaction mass may then be cooled to 90° C. and water added. The reaction mixture may be maintained at 90° C. for 60 min., whereupon further water may be added at such a rate as to maintain a solution and the resulting solution may be held at 80° C. for 30 min. and then slowly cooled to 20° C. over at least 90 min. The resulting slurry may be then aged at 20° C. for 30 min. The resulting slurry may be then cooled to 2° C. and aged at this temperature for at least 3.0 h. The suspension may be filtered and washed with additional water. The washed suspension may be used directly to produce the compound of formula II, i.e. without a separate isolation step.

In an embodiment, the present invention provides a process for preparing a compound of formula BA as shown below.

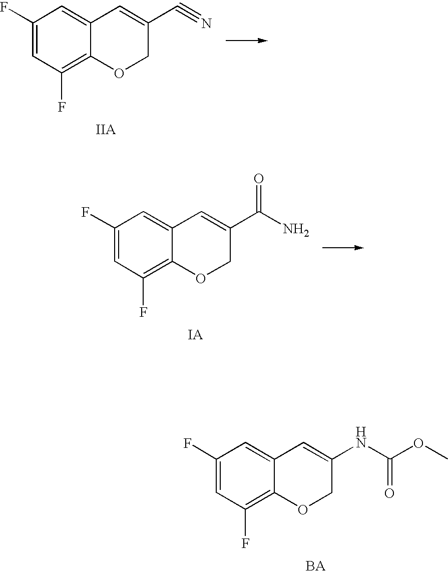

More particularly, the process of the present invention may involve the following steps:

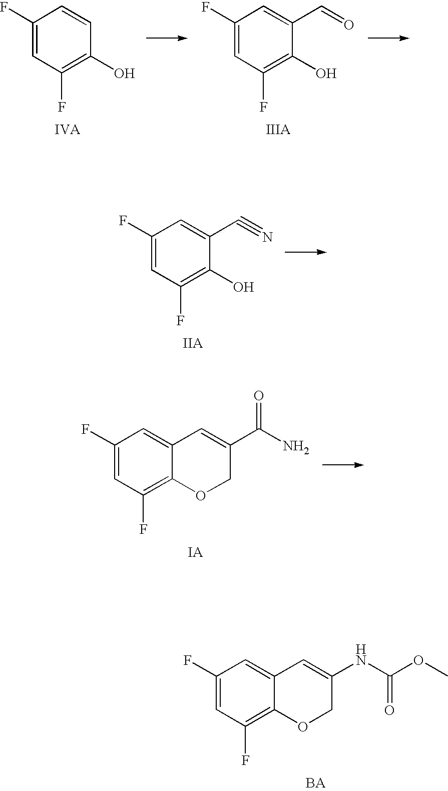

According to another aspect of the present invention, there is provided a process for preparing a compound of formula B:

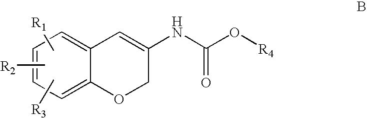

which process comprises converting a compound of formula V

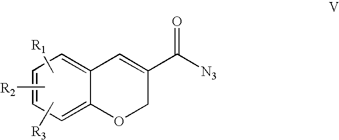

to the compound of formula B, wherein R₁, R₂ and R₃ are the same or different and signify hydrogens, halogens, alkyl, alkyloxy, hydroxy, nitro, alkylcarbonylamino, alkylamino or dialkylamino group; and R₄ is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

In an embodiment, at least one of R₁, R₂ and R₃ is fluorine. In another embodiment, compound V has the following formula:

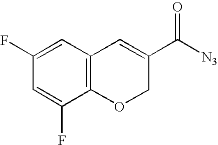

In an embodiment, R₄ is C₁ to C₄ alkyl. Optionally, R₄ is methyl, ethyl or t-butyl. Preferably, R₄ is methyl. In an alternative embodiment, R₄ is benzyl.

In an embodiment, the process is depicted as follows.

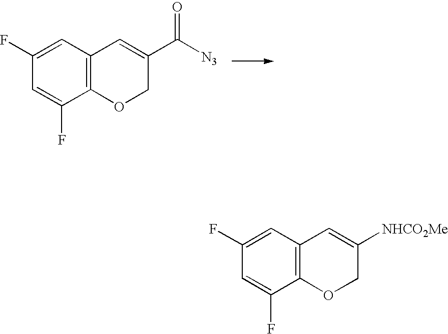

The conversion of V to B may involve thermal decomposition in the presence of an alcohol having the formula R₄OH, wherein R₄ has the same meanings as given above. In an embodiment, the thermal decomposition involves a Curtius rearrangement. The thermal decomposition may involve dissolving the compound of formula V in an organic solvent and heating the reaction mixture to the reflux temperature of the organic solvent. Suitable solvents include any substantially inert organic solvent, for example dichloromethane, toluene or ethyl acetate. Alternatively, the alcohol having the formula R₄OH can be used as the solvent as well as the reagent. The dissolution of the compound of formula V in the organic solvent may take place at an elevated temperature, for example at a temperature ranging from 35° C. to 80° C., preferably 50° C. to 70° C., preferably at a temperature of around 60° C.

After reaction completion, the reaction mixture may be cooled, optionally concentrated and a second organic solvent added to crystallise the compound of formula B. The second organic solvent may be any saturated hydrocarbon solvent, for example petroleum ether, hexane, or heptane. If the first organic solvent is water miscible, water may be added to crystallise the compound of formula B. The cooling may be to a temperature of less than 30° C., preferably less than 15° C.

It will be appreciated that the oxygen atom in the chromanyl ring may be replaced with a CH₂ group or a S atom such that the ring structure is a naphthalenyl ring or a thiochromanyl ring, respectively, and the conversion of V to B be carried out in the same manner as described above in relation to the chromanyl ring.

According to another aspect of the present invention, there is provided a process for preparing a compound of formula V, as defined above. The process involves converting a compound of formula VI:

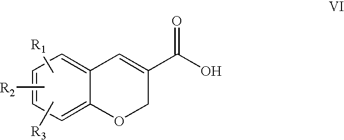

to the compound of V, wherein R₁, R₂, and R₃ have the same meanings as given above.

In an embodiment, at least one of R₁, R₂ and R₃ is fluorine. In an embodiment, the compound of formula VI has the formula VIA:

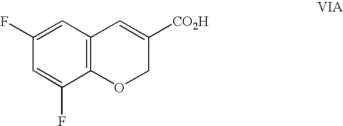

The conversion of VI to V may involve use of an acyl azide forming reagent, examples of which are well known to those skilled in the art, typically in the presence of a water miscible solvent, and optionally a base. Water may also be present.

The acyl azide forming reagent may be diphenyl phosphoryl azide in the presence of a base. The water miscible solvent may be acetone, acetonitrile, DMF, THF, dioxane or 1,2-dimethoxyethane. The base is preferably a weak base and may be triethylamine, tripropylamine or tributylamine.

The compound of formula V may be precipitated from the reaction mixture, for example by addition of cold water thereto. The suspension may then be cooled, filtered and the damp filter cake extracted with a suitable organic solvent. The solution of compound V in the extraction organic solvent may be taken directly for the conversion to B as discussed above, i.e. without a separate isolation step.

It will be appreciated that the oxygen atom in the chromanyl ring may be replaced with a CH₂ group or a S atom such that the ring structure is a naphthalenyl ring or a thiochromanyl ring, respectively, and the conversion of VI to V be carried out in the same manner as described above in relation to the chromanyl ring.

According to another aspect of the present invention, there is provided a process for preparing a compound of formula VI, as defined above. The process involves converting a compound of formula II:

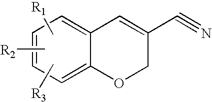

to the compound of formula VI, wherein R₁, R₂, and R₃ have the same meanings as given above.

In an embodiment, at least one of R₁, R₂ and R₃ is fluorine. In an embodiment, the compound of formula II has the formula IIA.

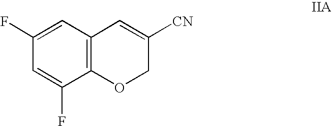

The conversion of II to VI may involve hydrolysing the carbonitrile having the formula II. The hydrolysis may involve reaction of the compound of formula II with a base, such as sodium hydroxide, lithium hydroxide or potassium hydroxide, in the presence of water, followed by a work-up with an acid, such as hydrochloric acid, sulphuric acid or phosphoric acid.

It will be appreciated that the oxygen atom in the chromanyl ring may be replaced with a CH₂ group or a S atom such that the ring structure is a naphthalenyl ring or a thiochromanyl ring, respectively, and the conversion of II to VI be carried out in the same manner as described above in relation to the chromanyl ring.

The compound of formula II may be prepared according to the process described above, i.e. by converting a compound of the formula III:

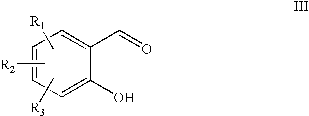

to the compound of formula II, wherein R₁, R₂ and R₃ have the same meanings as given above.

The compound of formula III may be prepared according to the process described above, i.e. by converting a compound of formula IV:

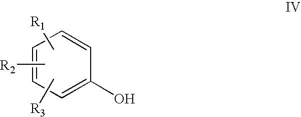

to the compound of formula III, wherein R₁, R₂ and R₃ have the same meanings as given above.

In an embodiment, the present invention provides a process for preparing a compound of formula B as shown below.

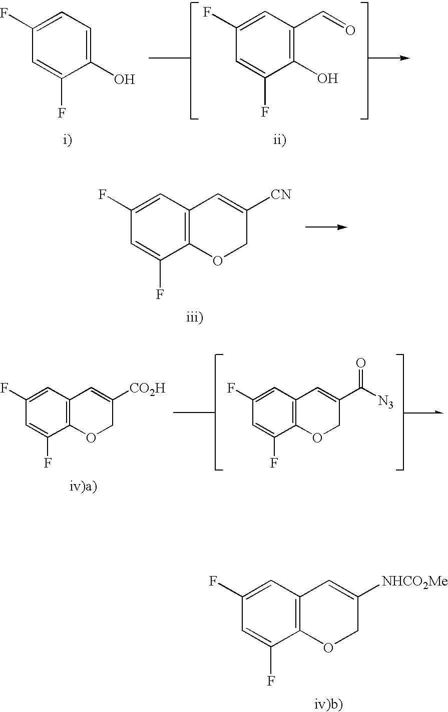

Suitably, the reaction conditions for the above steps are:
(i) Trifluoroacetic acid, hexamethylenetetramine, 80° C. then 115° C., water;
(ii) Dimethylformamide, acrylonitrile, 1,4-diazabicyclo[2.2.2]octane, water, 70° C.;
(iii) (a) Sodium hydroxide, water, 95° C.; (b) conc. hydrochloric acid;
(iv) (a) Acetone, triethylamine, diphenylphosphoryl azide, water; (b) dichloromethane, methanol, 60° C., petroleum ether.

All the steps in the processes of the present invention are safe and economical and result in good yields of product.

In an embodiment, the compound of formula B prepared according to any one of the processes of the present invention is converted to a compound of formula E:

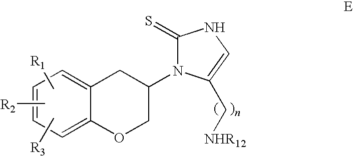

wherein R₁₂ signifies hydrogen, alkyl or alkylaryl group; n is 1, 2 or 3; and R₁, R₂ and R₃ have the same meanings as given above. The compound of formula E may be a compound having the formula P.

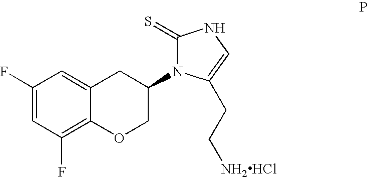

The conversion may involve the following steps. The compound of formula B is converted to the S or R enantiomer of a compound of formula A,

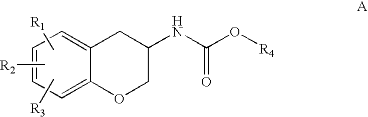

wherein R₁, R₂, R₃ and R₄ have the same meanings as given above.

In an embodiment, at least one of R₁, R₂ and R₃ is fluorine. Suitably, compound A has the following formula:

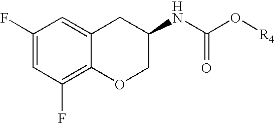

In an embodiment, R₄ is Cᵢ to C₄ alkyl. Optionally, R₄ is methyl, ethyl or tBu. Preferably, R₄ is methyl. In an alternative embodiment, R₄ is benzyl.

In an embodiment, compound A is in the form of the S enantiomer. In an alternative embodiment, compound A is in the form of the R enantiomer.

The R or S enantiomer of compound A may be converted to the respective R or S enantiomer of a compound of formula C, or a salt thereof.

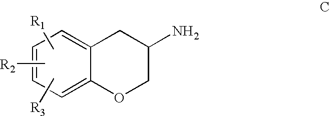

wherein R₁, R₂, and R₃ have the same meanings as given above. The R or S enantiomer of the compound of formula C, or a salt thereof, may be converted to the respective R or S enantiomer of a compound of formula E or a salt thereof

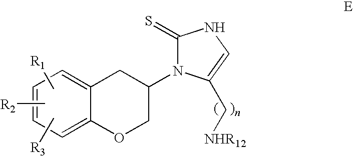

wherein R₁, R₂, and R₃ have the same meanings as given above; R₁₂ signifies hydrogen, alkyl or alkylaryl group; and n is 1, 2 or 3.

Preferably, E is (R)-5-(2-aminoethyl)-1-(6,8-difluorochroman-3-yl)-1,3-dihydroimidazole-2-thione.

In an embodiment, the R or S enantiomer of the compound of formula C is reacted with a compound of formula D2:

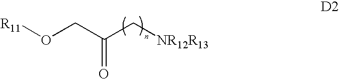

to produce the respective R or S enantiomer of a compound of formula E or a salt thereof

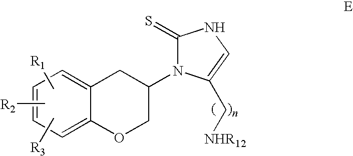

where R₁, R₂ and R₃ have the same meanings as given above n signifies 1, 2 or 3; R₁₂ signifies hydrogen, alkyl or alkylaryl group, R₁₁ signifies a hydroxyl protecting group and R₁₃ signifies an amino protecting group, or R₁₁ is defined as above but R₁₂ and R₁₃ taken together represent a phthalimido group; with a water soluble thiocyanate salt in the presence of an organic acid in a substantially inert solvent, followed by subsequent deprotection of the intermediate products F to I:

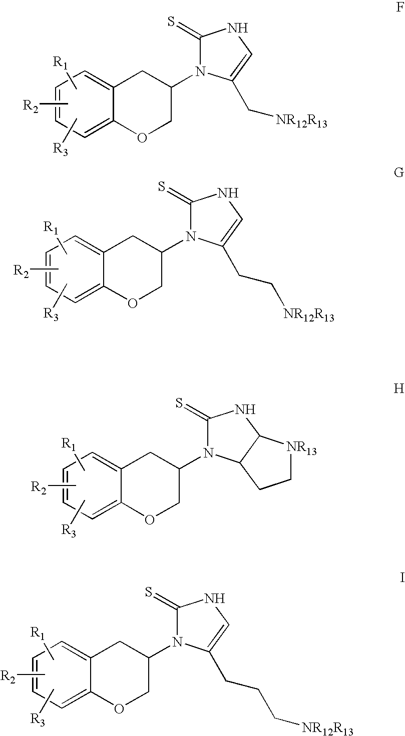

Preferably, the water soluble thiocyanate salt is an alkali metal thiocyanate salt or a tetraalkylammonium thiocyanate salt. Preferably, the solvent is an organic solvent.

In an embodiment, n is 2 or 3. In a further embodiment, at least one of R₁, R₂ and R₃ is fluorine. Optionally, the compound of formula E is:
(S)-5-(2-aminoethyl)-1-(1,2,3,4-tetrahydronaphthalen-2-yl)-1,3-dihydroimidazole-2-thione;
(S)-5-(2-aminoethyl)-1-(5,7-difluoro-1,2,3,4-tetrahydronaphthalen-2-yl)-1,3-dihydroimidazole-2-thione;
(R)-5-(2-aminoethyl)-1-chroman-3-yl-1,3-dihydroimidazole-2-thione;
(R)-5-(2-aminoethyl)-1-(6-hydroxychroman-3-yl)-1,3-dihydroimidazole-2-thione;
(R)-5-(2-aminoethyl)-1-(8-hydroxychroman-3-yl)-1,3-dihydroimidazole-2-thione;
(R)-5-(2-aminoethyl)-1-(6-methoxychroman-3-yl)-1,3-dihydroimidazole-2-thione;
(R)-5-(2-aminoethyl)-1-(8-methoxychroman-3-yl)-1,3-dihydroimidazole-2-thione;
(R)-5-(2-aminoethyl)-1-(6-fluorochroman-3-yl)-1,3-dihydroimidazole-2-thione;
(R)-5-(2-aminoethyl)-1-(8-fluorochroman-3-yl)-1,3-dihydroimidazole-2-thione;
(R)-5-(2-aminoethyl)-1-(6,7-difluorochroman-3-yl)-1,3-dihydroimidazole-2-thione;
(R)-5-(2-aminoethyl)-1-(6,8-difluorochroman-3-yl)-1,3-dihydroimidazole-2-thione;
(S)-5-(2-aminoethyl)-1-(6,8-difluorochroman-3-yl)-1,3-dihydroimidazole-2-thione;
(R)-5-(2-aminoethyl)-1-(6,7,8-trifluorochroman-3-yl)-1,3-dihydroimidazole-2-thione;
(R)-5-(2-aminoethyl)-1-(6-chloro-8-methoxychroman-3-yl)-1,3-dihydroimidazole-2-thione;
(R)-5-(2-aminoethyl)-1-(6-methoxy-8-chlorochroman-3-yl)-1,3-dihydroimidazole-2-thione;
(R)-5-(2-aminoethyl)-1-(6-nitrochroman-3-yl)-1,3-dihydroimidazole-2-thione;
(R)-5-(2-aminoethyl)-1-(8-nitrochroman-3-yl)-1,3-dihydroimidazole-2-thione;
(R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione;
(R)-5-aminomethyl-1-(6-hydroxychroman-3-yl)-1,3-dihydroimidazole-2-thione;
(R)-5-(2-aminoethyl)-1-(6-hydroxy-7-benzylchroman-3-yl)-1,3-dihydroimidazole-2-thione;
(R)-5-aminomethyl-1-(6,8-difluorochroman-3-yl)-1,3-dihydroimidazole-2-thione;
(R)-5-(3-aminopropyl)-1-(6,8-difluorochroman-3-yl)-1,3-dihydroimidazole-2-thione;
(S)-5-(3-aminopropyl)-1-(5,7-difluoro-1,2,3,4-tetrahydronaphthalen-2-yl)-1,3-dihydroimidazole-2-thione;
(R,S)-5-(2-aminoethyl)-1-(6-hydroxythiochroman-3-yl)-1,3-dihydroimidazole-2-thione; (R,S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or (R)-1-chroman-3-yl-5-(2-methylaminoethyl)-1,3-dihydroimidazole-2-thione.

The compound of formula E may also be a salt of:
(S)-5-(2-aminoethyl)-1-(1,2,3,4-tetrahydronaphthalen-2-yl)-1,3-dihydroimidazole-2-thione;
(S)-5-(2-aminoethyl)-1-(5,7-difluoro-1,2,3,4-tetrahydronaphthalen-2-yl)-1,3-dihydroimidazole-2-thione;
(R)-5-(2-aminoethyl)-1-chroman-3-yl-1,3-dihydroimidazole-2-thione;
(R)-5-(2-aminoethyl)-1-(6-hydroxychroman-3-yl)-1,3-dihydroimidazole-2-thione;
(R)-5-(2-aminoethyl)-1-(8-hydroxychroman-3-yl)-1,3-dihydroimidazole-2-thione;
(R)-5-(2-aminoethyl)-1-(6-methoxychroman-3-yl)-1,3-dihydroimidazole-2-thione;
(R)-5-(2-aminoethyl)-1-(8-methoxychroman-3-yl)-1,3-dihydroimidazole-2-thione;
(R)-5-(2-aminoethyl)-1-(6-fluorochroman-3-yl)-1,3-dihydroimidazole-2-thione;
(R)-5-(2-aminoethyl)-1-(8-fluorochroman-3-yl)-1,3-dihydroimidazole-2-thione;
(R)-5-(2-aminoethyl)-1-(6,7-difluorochroman-3-yl)-1,3-dihydroimidazole-2-thione;
(R)-5-(2-aminoethyl)-1-(6,8-difluorochroman-3-yl)-1,3-dihydroimidazole-2-thione;
(S)-5-(2-aminoethyl)-1-(6,8-difluorochroman-3-yl)-1,3-dihydroimidazole-2-thione;
(R)-5-(2-aminoethyl)-1-(6,7,8-trifluorochroman-3-yl)-1,3-dihydroimidazole-2-thione;
(R)-5-(2-aminoethyl)-1-(6-chloro-8-methoxychroman-3-yl)-1,3-dihydroimidazole-2-thione;
(R)-5-(2-aminoethyl)-1-(6-methoxy-8-chlorochroman-3-yl)-1,3-dihydroimidazole-2-thione;
(R)-5-(2-aminoethyl)-1-(6-nitrochroman-3-yl)-1,3-dihydroimidazole-2-thione;
(R)-5-(2-aminoethyl)-1-(8-nitrochroman-3-yl)-1,3-dihydroimidazole-2-thione;
(R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione;
(R)-5-aminomethyl-1-(6-hydroxychroman-3-yl)-1,3-dihydroimidazole-2-thione;
(R)-5-(2-aminoethyl)-1-(6-hydroxy-7-benzylchroman-3-yl)-1,3-dihydroimidazole-2-thione;
(R)-5-aminomethyl-1-(6,8-difluorochroman-3-yl)-1,3-dihydroimidazole-2-thione;
(R)-5-(3-aminopropyl)-1-(6,8-difluorochroman-3-yl)-1,3-dihydroimidazole-2-thione;
(S)-5-(3-aminopropyl)-1-(5,7-difluoro-1,2,3,4-tetrahydronaphthalen-2-yl)-1,3-dihydroimidazole-2-thione;
(R,S)-5-(2-aminoethyl)-1-(6-hydroxythiochroman-3-yl)-1,3-dihydroimidazole-2-thione; (R,S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or
(R)-1-chroman-3-yl-5-(2-methylaminoethyl)-1,3-dihydroimidazole-2-thione. Preferably, the salt is the hydrochloride salt.

According to another aspect of the present invention, there is provided compound of formula I:

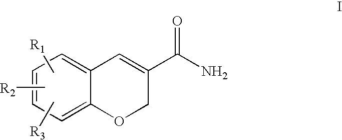

wherein R₁, R₂ and R₃ are the same or different and signify hydrogens, halogens, alkyl, alkyloxy, hydroxy, nitro, alkylcarbonylamino, alkylamino or dialkylamino group, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine. In an embodiment, at least one of R₁, R₂ and R₃ is fluorine. Suitably, compound I has the following formula IA.

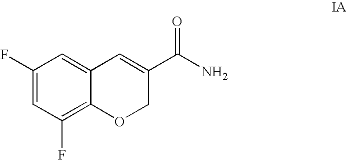

Compound I may be prepared by any suitable process, for example by any one of the processes described above.

According to another aspect of the present invention, there is provided a compound of formula II:

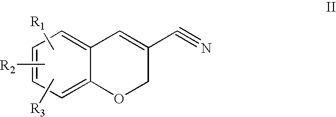

wherein R₁, R₂ and R₃ have the same meanings as given above. The compound of formula II may be prepared according to any one of the processes described above. Suitably, compound II has the following formula IIA.

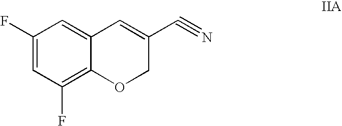

According to another aspect of the present invention, there is provided a compound of formula V:

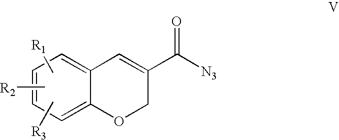

wherein R₁, R₂ and R₃ have the same meanings as given above. The compound of formula V may be prepared according to any one of the processes described above. Suitably, compound V has the following formula.

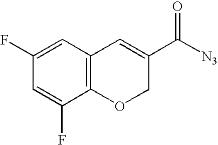

According to another aspect of the present invention, there is provided a compound of formula VI

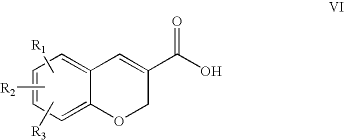

wherein R₁, R₂ and R₃ have the same meanings as given above. The compound of formula VI may be prepared according to any one of the processes described above. Suitably, compound VI has the following formula.

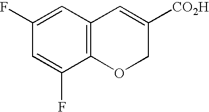

### EXAMPLES

The invention will now be described with reference to the following non-limiting examples.

### Example 1

### 6,8-difluoro-2H-chromene-3-carbonitrile—compound IVA to compound MA to compound IIA

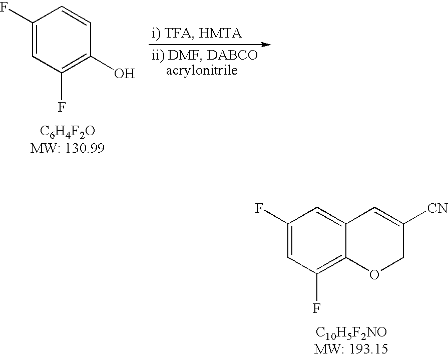

To a 100 L reactor was charged trifluoroacetic acid (11.25 L, 17.28 kg) and 2,4-difluorophenol (IVA, 2.25 kg); the resulting solution was adjusted to 20° C. With good stiffing hexamethylentetramine (2.70 kg) was charged over ˜30 minutes; the reaction temperature was allowed to attain 40° C. The reaction mixture was adjusted to 80° C. and held at 80° C. for at least 1.0 hour before heating to 115° C. The reaction mixture was held at 115° C. for 18.0 to 20.0 hours whereupon the reaction was cooled to 30° C. and the reactor charged with water (76.5 L) over at least 30 minutes. The reaction was then adjusted to 2° C. and held at 2° C. for at least 4.0 hours. The resulting suspension was then filtered and the filter cake washed twice with water (18.0 L and 13.5 L) and then pulled dry for at least 30 minutes.

Two lots of the water wet aldehyde (IIIA) were then employed in the following:

To a 100 L reactor was charged the water wet aldehyde (IIIA), acrylonitrile (7.9 kg), dimethyl formamide (13.5 kg) and water (18.5 L). With good stirring DABCO (0.88 kg) was added to affording a clear yellow solution. The reaction mixture was then adjusted to 70° C. and the reaction mixture was held at 70° C. for 18.0 to 20.0 hours, whereupon the reaction mixture was cooled to 20° C. Water (18.4 L) was then charged and the reaction mixture adjusted to 2° C. and held at 2° C. for 3 hours. The product was then filtered, washed with aqueous methanol (7.3 L) (5:1, MeOH:H₂O) and dried under vacuum at 45° C. to afford 6,8-difluoro-2H-chromene-3-carbonitrile (IIA, 2.90 kg, 43.5%) as a pale yellow crystalline solid.

### Example 1A

### 6,8-difluoro-2H-chromene-3-carbonitrile—compound IVA to compound MA to compound IIA

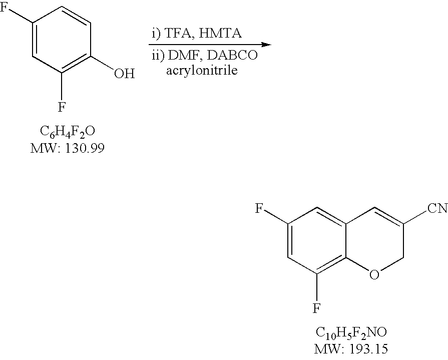

To a 100 L reactor was charged trifluoroacetic acid (20 L, 30.72 kg) and 2,4-difluorophenol (IVA, 4.0 kg); the resulting solution was adjusted to 20° C. With good stiffing hexamethylentetramine (4.80 kg) was charged over ˜30 minutes; the reaction temperature was allowed to attain 40° C. The reaction mixture was adjusted to 80° C. and held at 80° C. for at least 1.0 hour before heating to 115° C. The reaction mixture was held at 115° C. for 18.0 to 20.0 hours whereupon the reaction was cooled to 90° C. and the reactor charged with water (8 L). The reaction mixture was maintained at 90° C. for 60 min., then further water (52 L) was added at such a rate as to maintain a solution and the resulting solution was held at 80° C. for 30 min. and then slowly cooled to 20° C. over at least 90 min. The resulting slurry was then aged at 20° C. for 30 min. The resulting slurry was then cooled to 2° C. and aged at this temperature for at least 3.0 h. The suspension was then filtered and subsequently washed with additional water. (32 L and 24 L) and then pulled dry for at least 30 minutes.

To a 100 L reactor was charged the water wet aldehyde (IIIA), acrylonitrile (10.4 L)), dimethyl formamide (10.4 L)) and water (8 L). With good stirring DABCO (0.96 kg) was added to affording a clear yellow solution. The reaction mixture was then adjusted to 70° C. and the reaction mixture was held at 70° C. for 18.0 to 20.0 hours, whereupon the reaction mixture was cooled to 20° C. Water (20 L) was then charged over 20 min and the reaction mixture adjusted to 2° C. and held at 2° C. for 3 hours. The product was then filtered, washed with aqueous methanol (10 L) (2:1, MeOH:H₂O) and dried under vacuum at 45° C. to afford 6,8-difluoro-2H-chromene-3-carbonitrile (IIA, 3.64 kg, 61.3%) as a pale yellow crystalline solid.

### Example 2

### 6,8-difluoro-2H-chromene-3-carboxamide—compound HA to compound IA

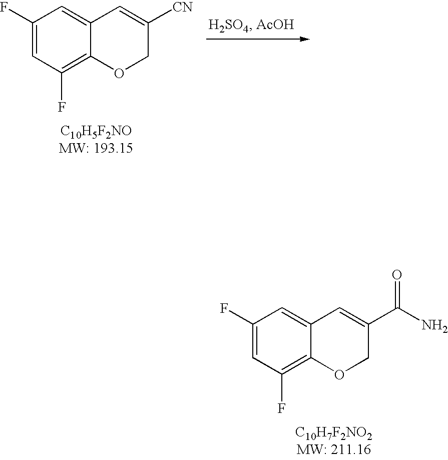

To a 100 L reactor was charged 6,8-difluoro-2H-chromene-3-carbonitrile (IIA, 2.86 kg) and acetic acid (22.9 L). With good stiffing the resulting suspension was adjusted to 20° C. whereupon sulphuric acid (10.96 kg) was charged in a single portion. The resulting suspension was then adjusted to 100° C. and maintained at 100° C. for 60 minutes. The reaction mixture was then adjusted to 30° C. and aqueous isopropanol (34.4 L (2:1, water:IPA)) charged over 20 minutes. The reaction mixture was then adjusted to 5° C. and held at 5° C. for at least 2.0 hours. The product was then filtered and the filter cake washed with aqueous isopropanol (14.3 L (2:1, water:IPA)), aqueous 0.5 N isopropanolic potassium hydroxide solution (12.0 L) and finally aqueous isopropanol (14.3 L (2:1, water:IPA)). The product was then dried under vacuum at 40° C. to afford 6,8-difluoro-2H-chromene-3-carboxamide (IA, 2.91 kg, 93.6%) as a microcrystalline solid.

### Example 2A

### 6,8-difluoro-2H-chromene-3-carboxamide—compound HA to compound IA

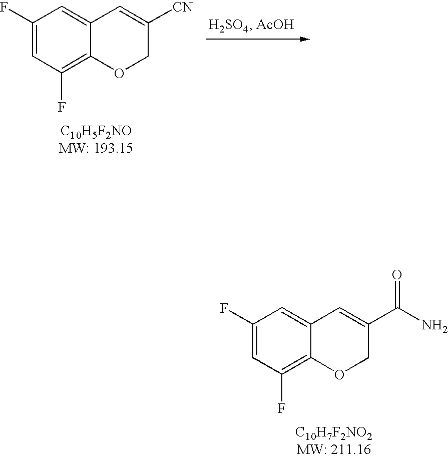

To a 100 L reactor was charged 6,8-difluoro-2H-chromene-3-carbonitrile (IIA, 4.30 kg) and acetic acid (34.4 L). With good stiffing the resulting suspension was adjusted to 20° C. whereupon sulphuric acid (16.47 kg) was charged in a single portion. The resulting suspension was then adjusted to 100° C. and maintained at 100° C. for 60 minutes. The reaction mixture was then adjusted to 30° C. and aqueous isopropanol (51.6 L (2:1, water:IPA)) charged over 20 minutes. The reaction mixture was then adjusted to 2° C. and held at 2° C. for at least 2.0 hours. The product was then filtered and the filter cake washed with cold aqueous isopropanol (2×21.5 L (2:1, water:IPA)). The product was then dried under vacuum at 40° C. to afford 6,8-difluoro-2H-chromene-3-carboxamide (IA, 4.42 kg, 93.9%) as a microcrystalline solid.

### Example 3

### Methyl 6,8-difluoro-2H-chromen-3-yl carbamate—compound IA to compound BA

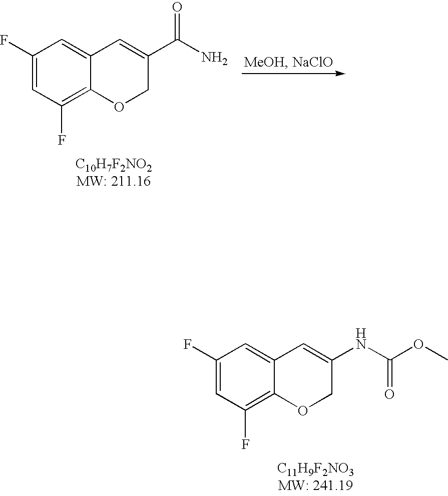

To a 100 L reactor was charged 6,8-difluoro-2H-chromene-3-carboxamide (2.88 kg) and methanol (44.7 L). With good stiffing the resulting suspension was adjusted to 5° C. whereupon aqueous sodium hypochlorite (8.25 L, 1.1 eq.) was charged at such a rate as to maintain the internal temperature below 10° C. The reaction mixture was then stirred at 5° C. for 30 minutes. The reaction mixture was sampled and analysed to confirm the complete consumption of the starting material. 1.5N sodium hydroxide solution (9.3 L) was then charged at such a rate as to maintain the internal temperature below 10° C. The reaction mixture was maintained at <10° C. for 30 minutes before adjusting the reaction mixture to 25° C. The reaction mixture was maintained at 25° C. for 20.0 to 24.0 hours. Whereupon the reaction mixture was adjusted to 5° C. before slowly charging 1.5N hydrochloric acid (20.0 L), the resulting suspension was maintained at 5° C. for at least 1.0 hour. The product was then filtered and washed with aqueous methanol (2×11.5 L (1:1, H₂O:MeOH)) and dried under vacuum at 45° C. to afford methyl 6,8-difluoro-2H-chromen-3-yl carbamate (2.45 kg, 74.5%) as a white microcrystalline solid.

### Example 3A

### Methyl 6,8-difluoro-2H-chromen-3-yl carbamate—compound IA to compound BA

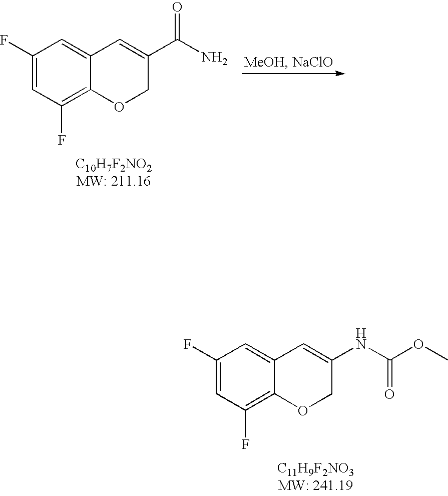

To a 100 L reactor was charged 6,8-difluoro-2H-chromene-3-carboxamide (3.1 kg) and methanol (48 L). With good stiffing the resulting suspension was adjusted to 5° C. whereupon aqueous sodium hypochlorite (8.3 L, 1.1 eq.) was charged at such a rate as to maintain the internal temperature below 10° C. The reaction mixture was then stirred at 5° C. for 30 minutes. The reaction mixture was sampled and analysed to confirm the complete consumption of the starting material. 1.5N sodium hydroxide solution (9.9 L) was then charged at such a rate as to maintain the internal temperature below 10° C. The reaction mixture was maintained at <10° C. for 30 minutes before adjusting the reaction mixture to 25° C. The reaction mixture was maintained at 25° C. for 20.0 to 24.0 hours. Whereupon the reaction mixture was adjusted to 5° C. before slowly charging water (21.7 L), the resulting suspension was maintained at 5° C. for at least 1.0 hour. The product was then filtered and washed with cold aqueous methanol (2×12.4 L (1:1, H₂O:MeOH)) and dried under vacuum at 45° C. to afford methyl 6,8-difluoro-2H-chromen-3-yl carbamate (2.62 kg, 74%) as a white microcrystalline solid.

### Re-crystallisation Procedure

To a 100 L reactor was charged water (9.1 L), 2-propanol (11.4 L) and methyl 6,8-difluoro-2H-chromen-3-yl carbamate (2.28 kg). With good stiffing the resulting suspension was adjusted to 75° C. and held at 75° C. until complete dissolution was achieved. The reaction mixture was then held at 75° C. for 30 minutes whereupon the reaction mixture was adjusted to 50° C. over 60 minutes and held at 50° C. for 60 minutes. The resulting suspension was then adjusted to 2° C. over 2.0 hours and held at 2° C. for at least 60 minutes. The product was then filtered and washed with aqueous 2-propanol (2×6.8 L (4:5, H₂O:IPA)) and dried under vacuum at 45° C. to afford methyl 6,8-difluoro-2H-chromen-3-yl carbamate (2.03 kg, 88.8%) as a white microcrystalline solid.

### Example 4

### 6,8-difluoro-2H-chromene-3-carboxylic acid—compound IIA to compound IVA

To a solution of sodium hydroxide (0.52 wt, 2.5 mol eq.) in water (14.0 vol.) at 20° C. was added 6,8-difluoro-2H-chromene-3-carbonitrile (1.0 wt) to afford a suspension. The reaction mixture is then heated to 95° C. and maintained at 95° C. until a clear solution is obtained. The reaction mixture is then monitored by HPLC until completion. The reaction mixture is then cooled to 20° C. and 36% hydrochloric acid (1.31 vol., 1.57 wt, 3.0 mol eq.) slowly added to afford a mobile suspension. The suspension is then cooled to <5° C. and maintained at <5° C. for at least 1.0 h. The title compound is then filtered and subsequently washed with additional water (2×2.0 vol.). The product is then dried under vacuum at 40° C. to constant weight.

### Example 5

### Methyl 6,8-difluoro-2H-chromen-3-yl carbamate—compound VIA to compound VA to compound BA

To a solution of 6,8-difluoro-2H-chromene-3-carboxylic acid (1.0 wt) in acetone (10.0 vol.) and triethylamine (0.71 vol., 1.09 mol eq.) at 15° C. was added diphenyl phosphoryl azide (1.1 vol., 1.09 mol eq.) in a single portion. The reaction mixture was then monitored by HPLC until completion. The reaction mixture was then diluted with cold water (20.0 vol.) to effect precipitation of the intermediate azide. The suspension was cooled to <10° C. and held at <10° C. for 1.0 h. The suspension was then filtered and subsequently washed with additional Water (5.0 vol.). The water wet material was then taken up into dichloromethane (7.5 vol.) and the resulting phases separated. The resulting dichloromethane solution was dried employing magnesium sulphate. The dichloromethane azide solution is then added to methanol (6.0 vol.) at 60° C. at such a rate that the rate of addition equals the collection of distillate. Upon full addition the distillation is continued until the distillate head temperature reaches 60° C. whereupon the system is set to reflux. The reaction is then monitored by HPLC until completion. The reaction mixture is then cooled to <15° C. and concentrated under vacuum to 2.0 vol. The crude reaction mixture is then diluted with dichloromethane (7.5 vol.) and heptane (2.5 vol.). The reaction mixture is then concentrated to 6.0 vol. via atmospheric distillation of dichloromethane. After cooling to 25° C. petroleum ether (10.0 vol.) is charge slowly to effect the crystallisation of the title compound. After full addition the resulting suspension is cooled to <5° C. and held at 5° C. for 1.0 h. The title compound is then filtered and washed with additional petroleum ether (5.0 vol.). The product is then dried under vacuum at 35° C. to constant weight.

It will be appreciated that the invention may be modified within the scope of the appended claims.

1. A process for preparing a compound of formula B: 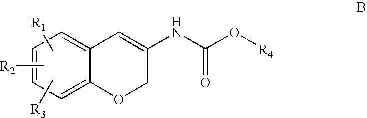
which process comprises converting a compound of formula VII: 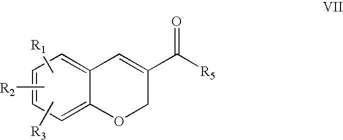
to the compound of formula B, wherein R₁, R₂ and R₃ are the same or different and signify hydrogens, halogens, alkyl, alkyloxy, hydroxy, nitro, alkylcarbonylamino, alkylamino or dialkylamino group; R₄ is alkyl or aryl; and R₅ is —N₃ or —NH₂, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

2. The process according to claim 1, wherein at least one of R₁, R₂ and R₃ is fluorine.

3. The process according to claim 1, wherein R₄ is C₁ to C₄ alkyl, preferably R₄ is methyl, ethyl or t-butyl, more preferably R₄ is methyl.

4-5. (canceled)

6. The process according to claim 1, wherein R₄ is benzyl.

7. The process according to any preceding claim 1, wherein compound VII has the formula I:

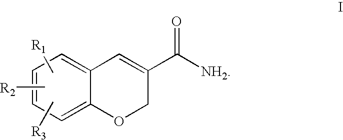

8. The process according to claim 1, wherein compound VII has the formula IA:

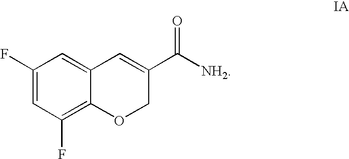

9. The process according to claim 1, wherein compound VII has the formula V:

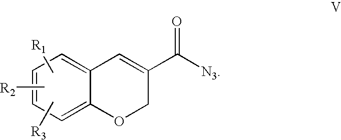

10. The process according to claim 1, wherein compound VII has the formula VA:

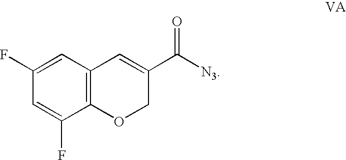

11. (canceled)

12. The process according to claim 7, wherein the conversion comprises a Hoffman rearrangement.

13. The process according to claim 9, wherein the conversion comprises a Curtius rearrangement.

14. The process according to claim 12, wherein the conversion comprises effecting a rearrangement of the amide group to the carbamate group in the presence of a hypohalite and an alcohol of the formula R₄OH, wherein R₄ is alkyl or aryl, preferably R₄ is C₁ to C₄ alkyl, preferably R₄ is methyl, ethyl or t-butyl, more preferably R₄ is methyl or benzyl.

15-17. (canceled)

18. The process according to claim 1, wherein the compound of formula B is purified by recrystallisation.

19. (canceled)

20. The process according to claim 7, wherein the compound of formula I is prepared by converting a compound of formula II: 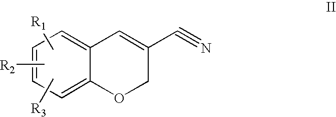
to the compound of I, wherein R₁, R₂ and R₃ have the same meaning as in compound I.

21-25. (canceled)

26. The process according to claim 1, wherein the compound of formula B has the following formula: 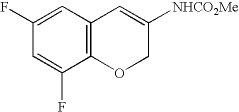
and the process for preparing the compound of formula B comprises the following steps:

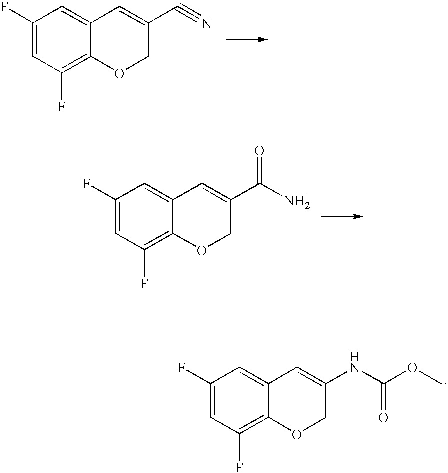

27. The process according to claim 13, wherein conversion of V to B comprises thermal decomposition in the presence of an alcohol having the formula R₄OH, wherein R₄ is C₁ to C₄ alkyl, preferably R₄ is methyl, ethyl or t-butyl, more preferably R₄ is methyl or benzyl.

28-37. (canceled)

38. The process according to claim 27, wherein the compound of formula V is prepared by converting a compound of formula VI: 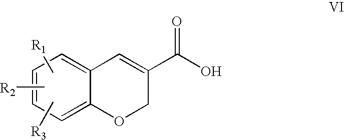
to the compound of V, wherein R₁, R₂, and R₃ have the same meanings as for compound V.

39-47. (canceled)

48. The process according to claim 38, wherein the compound of formula VI is prepared by converting a compound of formula II: 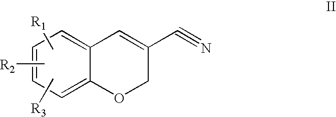
to the compound of formula VI, wherein R₁, R₂, and R₃ have the same meanings as compound VI.

49-53. (canceled)

54. The process according to claim 20, wherein the compound of formula II is prepared by converting a compound of the formula III: 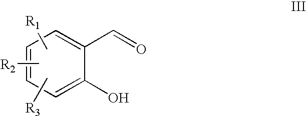
to the compound of formula II, wherein R₁, R₂ and R₃ have the same meanings as in compound II.

55-63. (canceled)

64. The process according to claim 54, wherein the compound of formula III is prepared by converting a compound of formula IV: 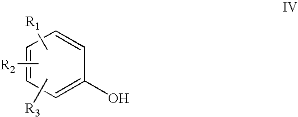
to the compound of formula III, wherein R₁, R₂ and R₃ have the same meanings as in compound III.

65-81. (canceled)

82. The process according to claim 64, wherein the compound of formula B has the following formula: 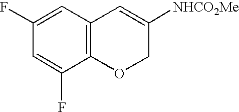
and the process for preparing the compound of formula B comprises the following steps:

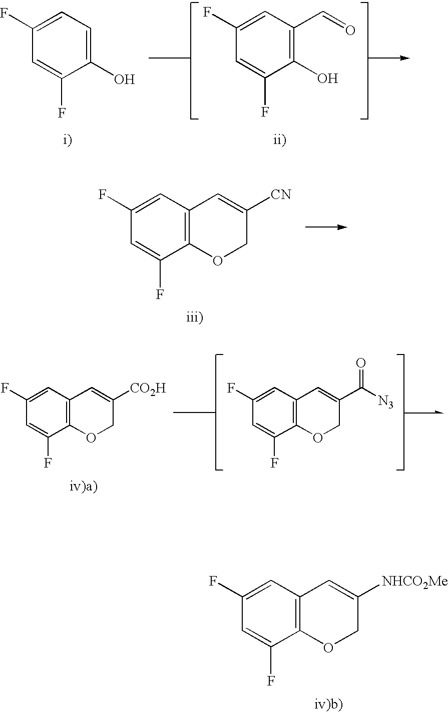

83. The process according to claim 64, wherein the compound of formula B has the following formula: 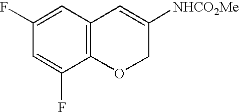
and the process for preparing the compound of formula B comprises the following steps:

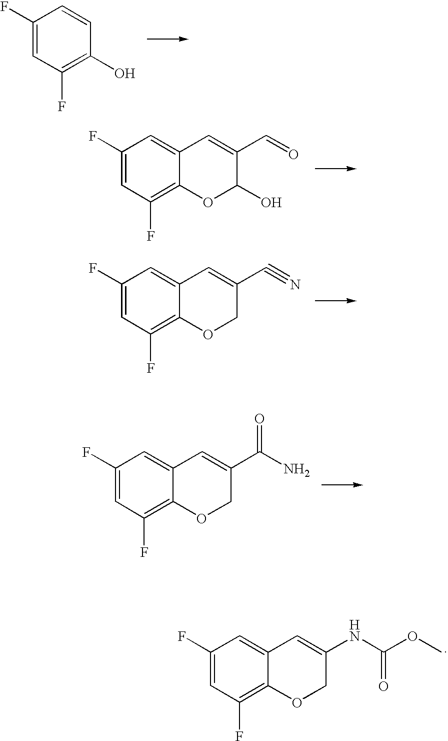

84. The process according to claim 1, wherein the compound of formula B is converted to the S or R enantiomer of a compound of formula A, 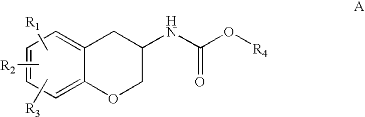
wherein R₁, R₂, R₃ are the same or different and signify hydrogens, halogens, alkyl, alkyloxy, hydroxy, nitro, alkylcarbonylamino, alkylamino or dialkylamino group, preferably at least one of R₁, R₂ and R₃ is fluorine and wherein R₄ is alkyl or aryl, preferably R₄ is C₁ to C₄ alkyl, preferably R₄ is methyl, ethyl or t-butyl, more preferably R₄ is methyl or benzyl.

85-91. (canceled)

92. The process according to claim 84, wherein the process further comprises converting the R or S enantiomer of compound A to the respective R or S enantiomer of a compound of formula C, or a salt thereof, 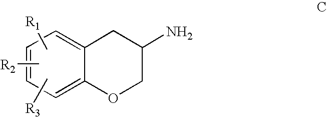
wherein R₁, R₂ and R₃ are the same or different and signify hydrogens, halogens, alkyl, alkyloxy, hydroxy, nitro, alkylcarbonylamino, alkylamino or dialkylamino group, wherein: the term alkyl means hydrocarbon chains, straight or branched, containing from one to six carbon atoms, optionally substituted by aryl, alkoxy, halogen, alkoxycarbonyl or hydroxycarbonyl groups; and the term halogen means fluorine, chlorine, bromine or iodine.

93. The process according to claim 92, wherein the R or S enantiomer of the compound of formula C, or a salt thereof, is converted to the respective R or S enantiomer of a compound of formula E or a salt thereof, 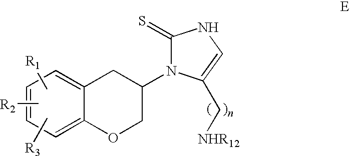
wherein R₁₂ signifies hydrogen, alkyl or alkylaryl group; and n is 1, 2 or 3.

94. The process according to claim 93, wherein the R or S enantiomer of the compound of formula C is reacted with a compound of formula D2: 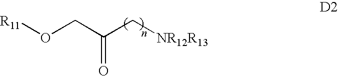
to produce the respective R or S enantiomer of a compound of formula E or a salt thereof, 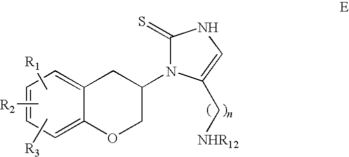
wherein n signifies 1, 2 or 3; R₁₂ signifies hydrogen, alkyl or alkylaryl group, R₁₁ signifies a hydroxyl protecting group and R₁₃ signifies an amino protecting group, or R₁₁ is defined as above but R₁₂ and R₁₃ taken together represent a phthalimido group; with a water soluble thiocyanate salt in the presence of an organic acid in a substantially inert solvent, followed by subsequent deprotection of the intermediate products F to I:

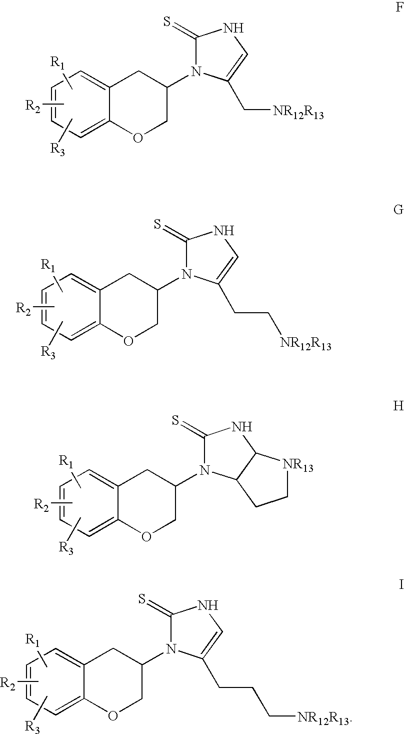

95-96. (canceled)

97. The process according to claim 94, wherein the compound of formula E is (R)-5-(2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluoro chroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluoro chroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3-dihydro imidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or (R)-1-chroman-3-yl-5-(2-methylaminoethyl)-1,3-dihydroimidazole-2-thione or a salt thereof.

98. (canceled)

99. The process according to claim 97, wherein the salt is the hydrochloride salt.

100. The process according to claim 94, wherein the compound of formula E is the R or S enantiomer of the compound of formula P.

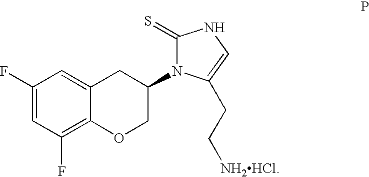

101. A compound of formula I: 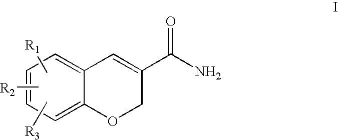
wherein R₁, R₂ and R₃ are the same or different and signify hydrogens, halogens, alkyl, alkyloxy, hydroxy, nitro, alkylcarbonylamino, alkylamino or dialkylamino group, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

102. (canceled)

103. A compound of formula V:

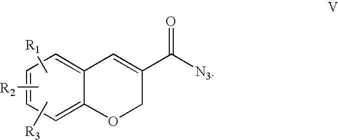

104. (canceled)

105. A compound of formula VI:

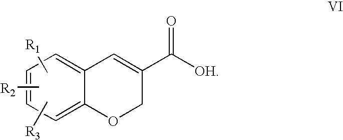

106. (canceled)

107. A compound of formula II:

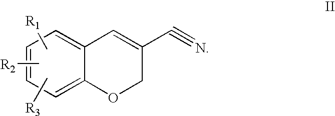

108-109. (canceled)
